(19) United States
(12) Patent Application Publication
(10) Pub. No.: US 2026/0035423 A1
(43) Pub. Date: Feb. 5, 2026

(54) Platelet lysate-based microparticles, methods and uses thereof

(71) Applicant: UNIVERSIDADE DE AVEIRO, AVEIRO (PT)

(72) Inventors: João Filipe COLARDELLE DA LUZ MANO, Aveiro (PT); Maria Clara FERREIRA DE ALMEIDA CARDIA GOMES, Aveiro (PT); Catarina DE ALMEIDA CUSTÓDIO, Aveiro (PT)

(73) Assignee: UNIVERSIDADE DE AVEIRO, AVEIRO (PT)

(21) Appl. No.: 19/139,428

(22) PCT Filed: Dec. 5, 2023
(86) PCT No.: PCT/IB2023/062242

§ 371 Date: Jun. 16, 2025

(30) Foreign Application Priority Data
Dec. 15, 2022 (PT) 118401

(51) Int. Cl. C07K 14/47; C07K 14/765; C07K 14/79; C12N 5/00

(52) U.S. Cl. CPC C07K 14/4741; C07K 14/765; C07K 14/79; C12N 5/0075

(57) ABSTRACT

The present disclosure relates to a process to assemble proteins derived from platelet lysates in bioactive microparticles, with increased surface organization. The present invention further relates to protein-based biomaterials applicable to biomedical and biotechnology fields, more precisely in tissue engineering strategies, disease modeling, and other biomedical applications. Namely, a method for obtaining a protein microparticle from a platelet lysate comprising the following steps: lyophilizing the platelet lysate; dissolving the lyophilized platelet lysate in phosphate buffer saline to obtain a platelet lysate solution; adding a reducing agent to the platelet lysate solution to obtain a reduced platelet lysate solution; adding an oxidizing agent to the reduced platelet lysate solution to form the microparticles by precipitation.

### TECHNICAL FIELD

The present disclosure relates to a process to assemble proteins derived from platelet lysates in bioactive microparticles, with increased surface organization. The present invention further relates to protein-based biomaterials applicable to biomedical and biotechnology fields, more precisely in tissue engineering strategies, disease modeling, and other biomedical applications.

### BACKGROUND

Microparticles are being explored as building blocks for bottom-up approaches, providing essential cues for bioengineering strategies. [1] In the last decades, microparticles have gained relevance for cell adhesion, expansion, and differentiation through the inclusion of biochemical or mechanical stimuli. Including microparticles in three-dimensional (3D) constructs can improve the diffusion of oxygen, nutrients, and chemical factors through the extracellular space of these constructs. [2] However, topographically textured microparticles are typically produced using synthetic polymers, which impairs their translation into clinics. [3]

Protein-based microparticles appear as an appealing alternative, encompassing the suitable biocompatibility, degradability, and biochemical signals for cells interaction. Proteins are emergent biomaterials with precise structure-function relationships boosting cell-cell interactions and cellular communications. [4] Currently, there are several procedures capable of producing protein-based microparticles, mostly arising through time consuming and expensive protocols (e.g., emulsions, lithography, electrospray, or microfluidics). [5]

Lai et al. reported a method for the fabrication of protein microparticles based on peptide mediated disulfide interchange reactions. The concept was based in using a redox reactive peptide as a natural crosslink reagent triggering the formation of intermolecular disulfide bonds between adjacent protein molecules. However, the assembly in a microparticle structure was dependent on a CaCO₃ template.

These facts are disclosed in order to illustrate the technical problem addressed by the present disclosure.

### General Description

This document discloses an invention related to the production of spherical microparticles based on platelet lysates with surface topography for applications in tissue engineering, and bioengineering.

The present disclosure provides the production of microparticles based on the assembly of proteins from platelet lysates, production processes, and uses thereof.

The present disclosure provides the controlled assembly of proteins recuring to the thiol groups present in their side chains residues through the creation of a reductive environment followed by an oxidative one.

In one embodiment, the invention comprises:
10-15% w/v of cell lysates;
1-10% of reducing agent;
1-5% of an oxidizing agent.

Specifically, the reducing agent can be mercaptoethanol, dithiothreitol, L-cysteine hydrochloride, cysteamine, or dithiobutylamine.

More specifically, the oxidizing agent can be hydrogen peroxide.

In one embodiment, the invention comprises protein-based microparticles produced via covalent crosslink using thiol groups creating of new disulfide bonds.

In the first aspect, the present disclosure relates to i) the use of disulfide bonds present in proteins and their reduction into thiol groups, or ii) by the insertion of new thiol groups through post-chemical modification of the proteins.

In a second aspect, the present disclosure relates to the assembly of the proteins via the formation of new disulfide bonds through oxidation.

In one embodiment, the present disclosure relates to protein-based microparticles with sizes between 10-100 μm.

In one embodiment, the present disclosure relates to protein-based microparticles with different shapes: round, star, or irregular.

In one embodiment, the present disclose relates to protein-based microparticles with surface topography with the lamellar microstructures spaced between 0.2-5 μm.

In one embodiment, the surface of the protein-based microparticles can be post-chemically modified with virtually any chemical moiety.

The present disclosure relates to bioactive microparticles as cell supporters for living cell culture.

The present disclosure relates to bioactive microparticles with defined surface topography for autonomous cell differentiation.

The present disclosure relates to an injectable system comprising the microparticles according to the present invention and cells.

The present disclosure relates to bottom-up strategies using the present invention and cells.

The present disclosure relates to a method for obtaining a protein microparticle from a platelet lysate comprising the following steps:
lyophilizing the platelet lysate;
dissolving the lyophilized platelet lysate in phosphate buffer saline to obtain a platelet lysate solution;
adding a reducing agent to the platelet lysate solution to obtain a reduced platelet lysate solution;
adding an oxidizing agent to the reduced platelet lysate solution to form the microparticles by precipitation.

In an embodiment for better results, the lyophilized platelet lysate is dissolved in phosphate buffer saline at a concentration ranging from 10 to 20% (w/v), preferably 12 to 16% (w/v).

In an embodiment for better results, the reducing agent is selected from a list comprising mercaptoethanol, dithiothreitol, L-cysteine hydrochloride, cysteamine, and dithiobutylamine.

In an embodiment for better results, the reducing agent is L-cysteine hydrochloride.

In an embodiment for better results, the concentration of the reducing agent ranges from 1.5%-10% (w/v).

In an embodiment for better results, the oxidizing agent is hydrogen peroxide.

In an embodiment for better results, the concentration of the oxidizing agent ranges from 1%-5% (w/v).

Another aspect of the present disclosure relates to a microparticle obtainable by the method described in any of the previous claims comprising a plurality of proteins from platelet lysate.

In an embodiment for better results, the microparticles comprise serum albumin, serotransferrin, keratin I, keratin II, or mixtures thereof.

In an embodiment for better results, the surface topography of the microparticles is tuneable. Namely by the addition of chaotropic or reducing agents.

In an embodiment for better results, the surface of the microparticle comprises a plurality of subunits, preferably with lamellar shape, and thicknesses between 0.1 μm and 0.2 μm, and spaced apart between 0.5 μm to 4 μm.

In an embodiment for better results, the size of the microparticle ranges from 10 to 100 μm, preferably from 20 to 50 μm.

In an embodiment for better results, the surface of the microparticle is chemically modified with a chemical moiety, preferably a fluorophore, a dye, a drug, an antibody, or combinations thereof.

Another aspect of the present disclosure relates to composition comprising the microparticle described in any of the previous claims.

In an embodiment for better results, the composition is an injectable composition.

In an embodiment, the composition may further comprise a plurality of cells, a therapeutic molecule, a suitable cell culture media, or mixtures thereof.

Another aspect of the present disclosure relates to the use of a microparticle as described in the present disclosure as a scaffold for cell adhesion, cell proliferation and/or cell differentiation.

Another aspect of the present disclosure relates to an article comprising the microparticle described in the present disclosure or the composition described in the present disclosure.

In an embodiment for better results, the article is a hydrogel, a fiber, a liquified capsule, a bioink, a microarray, or a lab-on-a-chip platform.

Another aspect of the present disclosure relates to the use of the microparticle of the present disclosure or a composition of the for use in the treatment of a bone defect, cartilage abrasion or myocardial infarction.

An aspect of the present disclosure relates to the use of a microparticle or a composition as described in the present disclosure for the manufacture of a medicament for the treatment of a bone defect, cartilage abrasion or myocardial infarction.

Another aspect of the present disclosure relates to a method for treating or preventing a bone defect, cartilage abrasion or myocardial infarction in a subject, the method comprising administering microparticle or a composition as described in the present disclosure.

### BRIEF DESCRIPTION OF THE DRAWINGS

The following figures provide preferred embodiments for illustrating the disclosure and should not be seen as limiting the scope of invention.

FIG. 1: Schematic representation of an embodiment of the production of protein-based microparticles from platelet lysates.

FIG. 2: (A) Characterization of the microparticle size according to the amount of the reducing agent (L-cysteine hydrochloride-L-Cys) used (1.5, 3, and 6% w/v), using 1% (v/v) of the oxidizing agent. The overall diameter of the microparticles can be related to the L-Cys concentration used. The microparticle diameter can be controlled by playing with the L-Cys concentration achieving diameters around 15 μm (for 1.5% L-Cys), 30 μm (for 3% L-Cys), and 50 μm (for 6% of L-Cys). (B) Concentration of proteins that contribute to the formation of the microparticles concerning the L-Cys concentration used. (C) Quantification of the free thiol groups using the Ellman's assay. The proteins are exposed to the same reducing and oxidizing environment used to produce the microparticles. (D) sodium dodecyl sulfate-polyacrylamide gel electrophoresis (SDS-PAGE) of the protein profile from the produced microparticles. L1: platelet lysates; L2: microparticles. The microparticle disintegration is performed using amounts of mercaptoethanol higher than the one suggested by the SDS-PAGE protocols.

FIG. 3: Morphological characterization of the protein-based microparticles by scanning electron microscopy (SEM). (A) The figure shows spherical microparticles with rough topography. (B) Close-up of the formed microparticles showing i) a laminated surface; ii) stacking of lamellar-like microstructures; iii) polyhedral-like microstructures that work as building units on the formation of the microparticles.

FIG. 4: Characterization of the topography of the microparticles surface after being treated with (A) chaotropic agent (calcium chloride-CaCl₂), 1 M) and (B) reducing agent (mercaptoethanol-ME, 6% v/v).

FIG. 5: (A) Live/Dead images of human-derived fibroblasts (hFB), human-derived umbilical vein endothelial cells (HUVECs), human-derived bone marrow stem cells (hBM-MSC), and mouse cardiomyoblasts (H9C2) seeded with the disclosed microparticles. Cells and microparticles were allowed to interact in flat ultra-low adhesion well plates for 2 days at 37° C. in cell culture media. (B) Fluorescent images of cells stained in orange (F-actin filaments) and blue (nuclei), and hPL microparticles in green, evidencing the adherence of the designated cells to the surface of the microparticles.

FIG. 6: (A) Optical images of the hBM-MSCs cells and H9C2 cells interacting with the microparticles. Identical amount of microparticles is seeded with different cell densities (25 000, 50 000, and 100 000 cells). Cells/microparticles are allowed to interact in flat ultra-low adhesion well plates for 2 days at 37° C. in cell culture media. (B) SEM images of cells/microparticle. Scale bar in μm.

FIG. 7: (A) Embodiment of fluorescence microscopy images from Live/Dead assay of hBM-MSCs cells seeded with the disclosed microparticles. Cells and microparticles were allowed to interact in flat ultra-low adhesion well plates for 21 days at 37° C. in cell culture media. (B) Cell metabolism of the cells/microparticles aggregates over 21 days at 37° C. in cell culture media. (C) DNA quantification of the cells/microparticles aggregates over 21 days at 37° C. in cell culture media. (D) Fluorescent images of cells stained in red (F-actin filaments) and blue (nuclei) evidencing the morphology of cells and F-actin organization over 21 days at 37° C. in cell culture media. (E) Histological sections from the cell/hPL microparticles aggregate stained with hematoxylin/eosin. Dark pink is observed as extracellular matrix (ECM) formation, with reddish pink as ECM condensation. Scale bar in μm.

FIG. 8: Images of the injectable system. A) Photograph of the cells/microparticles after injection into an agarose mold; B) Live/dead staining of cells/microparticles after injection observed by fluorescence microscopy; C) SEM imagens of the system cultured for 3 days at 37° C. showing the interaction between both elements. Scale bar in μm.

FIG. 9: Embodiment of autonomous differentiation of hBM-MSCs cells when in direct contact with the microparticles. A) Immunofluorescence of i-osteopontin and ii-osteocalcin markers in the cell/microparticles aggregates, and iii-histological section stained with Alizarin Red S. Calcium deposits are observed as dark spots in the formed microtissue; B) i) SEM micrograph, and ii) fluorescent image of a cell completely wrapping one PL microparticle. Scale bar in μm.

### DETAILED DESCRIPTION

The present disclosure relates to a process to assemble proteins derived from platelet lysates in bioactive microparticles, with increased surface organization. The present invention further relates to protein-based biomaterials applicable to biomedical and biotechnology fields, more precisely in tissue engineering strategies, disease modeling, and other biomedical applications.

The present disclosure relates to microparticles prepared from platelet lysate-derived components, processing methods, and uses thereof. In particular, the present disclosure relates to the use of these microparticles for cellular adhesion and proliferation. In particular, the present disclosure relates to the use of the disclosed microparticles as cell differentiators, without the need to had external chemical inductors of cell differentiation. In particular, the present disclosure relates to the use of the disclosed microparticles as injectable systems. In particular, the present disclosure relates to using these microparticles for bottom-up engineering tissues.

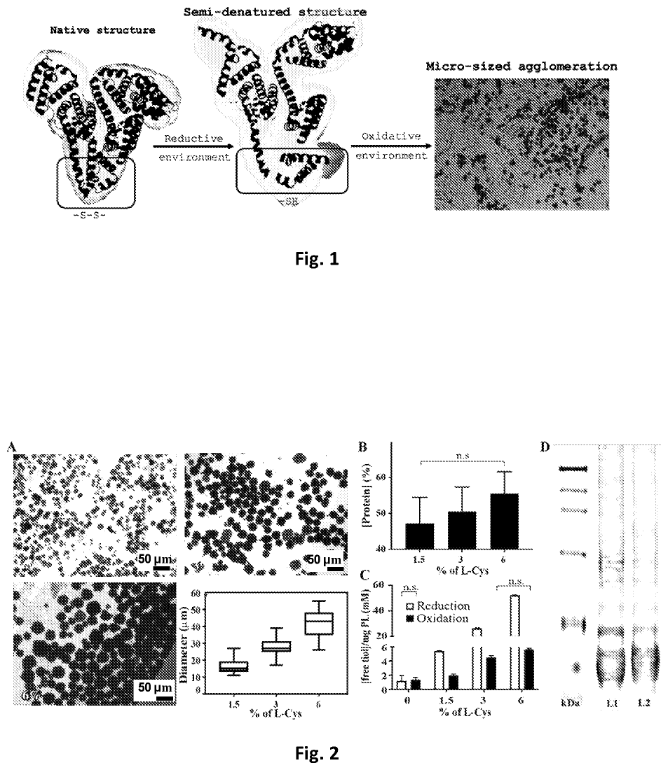

In an embodiment, schematically represented in FIG. 1, proteins need to have available thiol groups that can be achieved from native cysteine residues, reduced disulfide bonds, or prosthetic groups chemically inserted in the proteins side chains residues. The assembly of proteins is controlled by creating an oxidative environment, allowing the formation of new disulfide bonds between the same protein or between different proteins. Over time a white precipitate starts to appear, indicating the formation of the microparticles (micro-sized assembly).

In an embodiment, the microparticles are prepared from human-derived platelet lysates (hPL). Lyophilized hPL are dissolved in Phosphate Buffer Solution (PBS) at a final concentration of 14% (w/v).

In an embodiment, the reductive environment is created by adding L-cysteine (L-Cys) (1.5%-10% w/v, final concentration in the hPL solution) to the hPL solution. L-Cys is allowed to interact for 10 minutes. The existing disulfide bonds will be reduced at this point, creating free thiols groups.

In one embodiment, the oxidative environment is then created by adding hydrogen peroxide (H₂O₂, 1% v/v) to the previous mixture and allowed to interact for at least 3 h, forming a white precipitate (FIG. 1). The previously created thiol groups will be oxidized and form new disulfide bonds, assembling the proteins. These disulfide bridges can be formed within the same protein backbone or between two or more proteins.

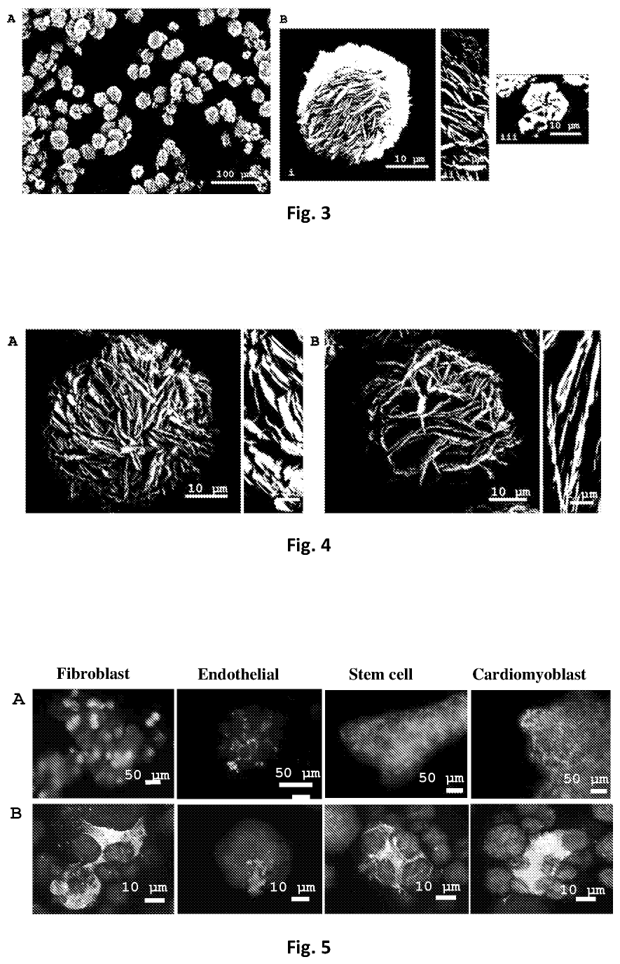

In one embodiment, the resulting precipitate is washed with PBS and centrifuged at 300 g for 5 min. The diameter of the hPL microparticles can be assessed from optical microscope images and using the Image processing program. hPL particle size varied from 20 μm (1.5% L-Cys), 30 μm (3% L-Cys), and 50 μm (6% L-Cys) (FIG. 2A). Bradford assay (FIG. 2B) can be used to quantify the protein that was involved in the microparticles formation, with bigger microparticles (in diameter) requiring more proteins; Ellman's assay (FIG. 2C) can be used to confirm the reduction/oxidation rate within the protocols followed (e.g., % L-Cys used). Higher concentrations of the reducing agent led to the reduction of a higher amount of disulfide bonds, increasing the amount of free thiols compared to the proteins not subjected to a reducing environment (% of L-Cys used). When the oxidizing agent is introduced, the existing free thiol groups decrease with the formation of new disulfide bonds; and sodium dodecyl sulfate-polyacrylamide gel (SDS-PAGE) can be used to identify by molecular weight the proteins (FIG. 2D) composition of the final microparticles, with Serum Albumin, Serotransferrin, and Keratin (I and II) being the most abundant proteins. The structural characterization of the hPL microparticles by scanning electron microscopy (SEM) reveals that these microparticles are round-shaped and well dispersed (FIG. 3A). Moreover, they are organized by 2D lamellar micro-sized subunits that assemble in 3D round-shaped structures (FIG. 3B). In a further embodiment, the surface of the microparticle comprises a plurality of subunits, preferably with lamellar shape, and thicknesses between 0.1 μm and 0.2 μm, and spaced apart between 0.5 μm to 4 μm.

In one embodiment, the surface topography of the hPL microparticles is personalized by incubating them with a chaotropic agent (e.g., calcium chloride (CaCl₂)), 1 M) or a reducing agent (e.g., mercaptoethanol (ME) 6% w/v) at room temperature (RT) for up to 2 days. The microparticles are then washed several times with PBS. The structure of post-changed topography (CaCl₂) and ME) was analyzed by SEM, revealing different topographies from pristine microparticles (original protocol) differing in the organization of the subunits that comprise the microparticles (FIGS. 4A and B). The use of CaCl₂) allows the modulation of the surface topography by increasing the space between each lamellar-like building block. In turn, the use of ME will further improve this distance and decrease the lamella thickness.

For the scope and interpretation of the present disclosure it is defined that “room temperature” should be regarded as a temperature between 15-30° C., preferably between 18-25° C., more preferably between 20-22° C.

In an embodiment, the surface of the hPL-based microparticles can be chemically post-modified with virtually any chemical moiety (e.g., fluorophores, dyes, drugs, antibodies, among others).

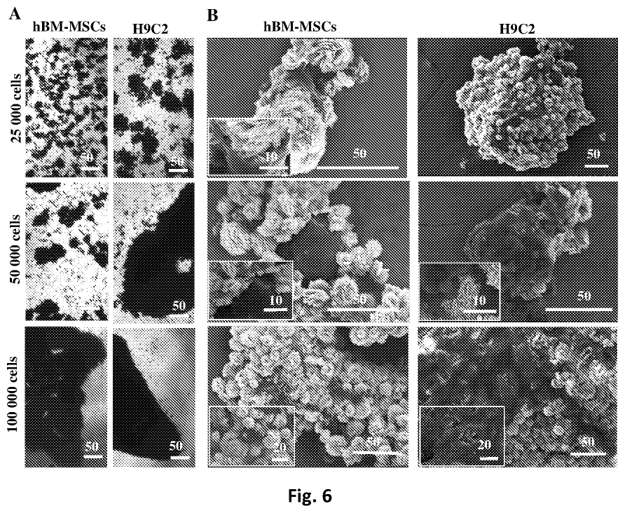

In an embodiment, the biocompatibility of these microparticles was evaluated towards cells from different origins (e.g., skin, bone marrow, veins, heart). Each cell line was seeded with hPL microparticles in flat ultra-low adhesion microplates using a culture media suitable to each cell and let to interreact for up to 2 days at 37° C. A live/dead fluorescence assay was used to evaluate the live cells. The cell/microparticles aggregates were incubated with calcein AM (staining the live cells in green), and propidium iodide (staining dead cells in red) for 30 min at 37° C. Results (FIG. 5A) show that most of the cells remained viable. The stem cells (hBM-MSC cells) and the cardiomyoblasts (C9H2 cells) formed large cell/microparticles aggregates after interaction with the surface of the microparticles. The agglomeration of cells and microparticles indicates an active interaction between both players.

In an embodiment, the cell/microparticles interaction was further evaluated by fluorescent microscopy. Cells are permeabilized with Triton-X100 (0.1% v/v in PBS) for 5 min, the cytoskeleton stained with red phalloidin (1:500), and the nucleus with DAPI (4′,6-diamidino-2-phenylindole dihydrochloride, 1:1000) (FIG. 5B). The presence of actin filaments shows a strong interaction of the cells with the surface of the microparticles. Cells can fully cover one microparticle or adhere to several ones enabling their spread. Cells can interact with more than one microparticle at the same time, showing strong actine filaments.

In an embodiment, different cell densities (25,000, 50,000, and 100,000 cells) are mixed with the same amount of hPL microparticles (˜250,000 microparticles) in a flat bottom 96-well ultra-low adhesion plates. After 2 days in culture, optical microscopy showed that cell density is a determinant parameter for the size of the formed cell/microparticles aggregates (FIG. 6A). After that, the aggregates are fixed with formaldehyde solution (4% v/v in PBS) for 30 min at RT, dehydrated in increasing ethanol series for 10 min at RT and incubated overnight in hexamethyldisilazane. Samples were coated with gold/palladium and visualized by scanning electron microscopy (SEM). with cells working as a glue to create the 3D construct (FIG. 6B). Indeed, several aspects can be evidenced: i) formation of autonomous aggregates without the need to apply external forces such as centrifugation; ii) aggregates sizes can be controlled by the cell density used. SEM images of cells/microparticle aggregate showed that cells can proliferate throughout the aggregates.

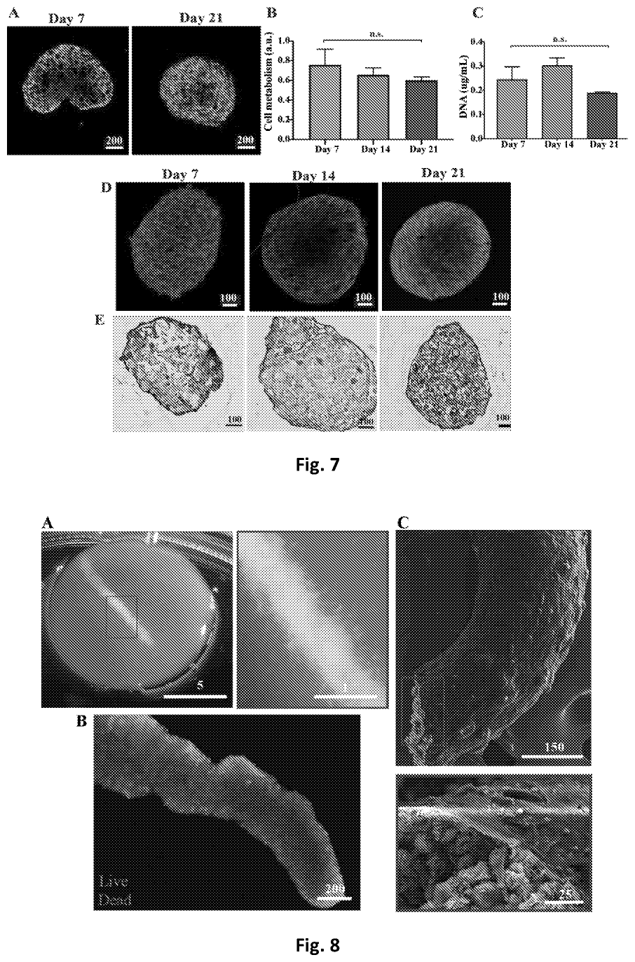

In an embodiment, stem cells (hBM-MSCs cells) are mixed (50, 000 cells and ˜250,000 microparticles) in a flat bottom 96-well ultra-low adhesion plates and cultured in a suitable media for 21 days. The viability of the cell/microparticles aggregates are assessed by the live/dead fluorescence assay (FIG. 7A) showing that most of the cells are alive for up to at least 21 days. An MTS colorimetric assay (FIG. 7B) was used to assess the cellular metabolism. Cell/microparticle aggregates were incubated with a MTS solution (1:6 in cell culture media) and incubated for 4 h at 37° C. The absorbance was read at 490 nm using a microplate reader, showing no significant difference in the cellular metabolism over the 21 days in culture. The Quanti-iT PicoGreen dsDNA assay kit was used for DNA quantification (FIG. 7C). The cell/microparticle aggregates were washed with MiliQ water, homogenized using an overhead stirrer for cell lysis, and centrifuged (6000 rpm) to collect the dsDNA supernatant. The supernatant and the PicroGreen reagent were mixed (1:1, v/v) and incubated for 10 min at RT. Fluorescence was read (Ex: 485/20 nm, Em: 528/20 nm) using a microplate reader, with results showing no significant difference over the 21 days in culture. The morphology of the cells/microparticle aggregates was also evaluated by fluorescent microscopy (FIG. 7D). Cells are permeabilized with Triton-X100 (0.1% v/v in PBS) for 5 min, the cytoskeleton stained with red phalloidin (1:500), and the nucleus with DAPI (4′,6-diamidino-2-phenylindole dihydrochloride, 1:1000). The actin filaments show cell morphology changing over the 21 days, with evident elongation and alignment from day 24 revealing the capability of cells to spread. Histology staining with hematoxylin/eosin (H&E) is done to the cell/microparticles aggregates to see their interior organization (FIG. 7E). The cell/microparticle aggregates were prefixed with paraformaldehyde (4%, v/v) for 30 min at RT, and inserted in HistoGel™ Specimen Processing Gel, dehydrated, and embedded in paraffin. Histological sections of 5 μm were performed using a microtome, and slices placed in adhesive slides, deparaffinized. Results show the formation of extracellular matrix (ECM) in dark pink, with the formation of reddish pink bodies as ECM condensation. Moreover, the aggregates present a loosed architecture with the presence of void spaces (without the presence of cells or hPL microparticles), that compact over time. The morphology of the hPL microparticles also change over time inside the aggregates.

In an embodiment, an injectable system is created by using a combination of microparticles and cells dispersed in cell culture media. The resulting formulation can then be transferred to a syringe and injected. In an embodiment, the resulting formulation was injected in an agarose mold using a 21 G needle (FIG. 8A). After 2 days in culture, the injectable system is visualized with a live/dead fluorescence assay (FIG. 8B), showing most of the cells live. The injected system was then extruded from the agarose mold, dehydrated and analyzed by SEM (FIG. 8C) showing the interaction between cells and microparticles displaying the organization into a round-shaped filament. As with the aggregates, cells wrap the microparticles forming a structure with the same shape as the injection site (i.e., the simulation of a defect). Thereafter, cells start to spread along the surface creating a monolayer.

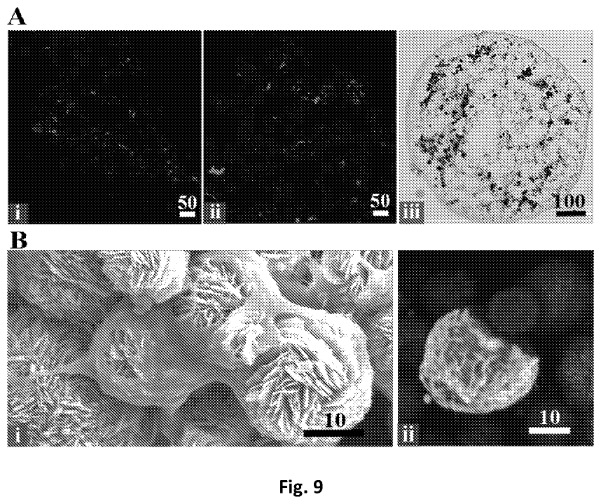

In an embodiment, the autonomous differentiation of the hBM-MSCs cells is evaluated by interacting cells with the pristine microparticles (e.g., with the original surface topography). hBM-MSCs/microparticles are cultured in a suitable media without any osteogenic supplementation for up to 21 days. The formed aggregates are tested for the expression of well-known bone differentiation markers, osteopontin (FIG. 9A-i) and osteocalcin (FIG. 9A-ii), and alizarin red S (FIG. 9A-iii). The expression of late bone markers, and presence of calcium deposits inside the aggregates without osteogenic inductors, indicate the autonomous differentiation of hBM-MSCs cells in osteoblasts, and the mineralization of the produced extracellular matrix. Cells can adhere to the microparticles completely wrapping them (FIG. 9B-i), presenting actin filaments oriented with the topography of the hPL microparticles (FIG. 9B-ii). This shows that cells can sense topography, which may lead to the autonomous cell differentiation.

The disclosed microparticles can be used as a component of hydrogels, fibers, liquified capsules, or bioinks to improve mechanical support and/or cell adhesion and proliferation.

In an embodiment, to the disclosed microparticles, produced from human-derived platelet lysates, provide an appropriate microenvironment for cells to adhere and proliferate. Differently from other protein-derived microparticles, the increased stability and tunable surface topographies of the disclosed microparticles makes it more suitable for multiple applications. The fine-tune the surface topography can confer microparticles with mechanical properties suitable to target specific tissues. Besides that, the present described microparticles can be combined with other materials and/or bioactive factors to enhance its biochemical and mechanical properties, which also enhances the range of possible applications, such as a delivery matrix or a graft material.

In an embodiment, the disclosed microparticles finds applicability for several purposes. In an embodiment, the disclosed microparticles can be used as a cell culture platform. For such purpose, the microparticles can be placed in ultra-low adhesion multi-well plates along with the desired cells.

In another embodiment, is the disclosed microparticles can be used as an injectable system. An injectable system can be used alone or combined with cells or therapeutic molecules. For such purpose, the microparticles may be injected into the patient at the site of injury or defect for regenerating/treating an injured site, such as a bone defect, cartilage abrasion, myocardial infarction.

In yet another embodiment, the disclosed microparticles can be incorporated into microfluidic, microarray, or lab-on-a-chip platforms.

All approaches can serve in vitro studies of multiple fields (e.g., pharmaceutic, tissue engineering, biotechnology, disease models) or commercial purposes (e.g., cell expansion, growth factors production).

The term “comprising” whenever used in this document is intended to indicate the presence of stated features, integers, steps, components, but not to preclude the presence or addition of one or more other features, integers, steps, components or groups thereof.

It will be appreciated by those of ordinary skill in the art that unless otherwise indicated herein, the particular sequence of steps described is illustrative only and can be varied without departing from the disclosure. Thus, unless otherwise stated the steps described are so unordered meaning that, when possible, the steps can be performed in any convenient or desirable order.

The disclosure should not be seen in any way restricted to the embodiments described and a person with ordinary skill in the art will foresee many possibilities for modifications thereof. The above-described embodiments are combinable.

The following claims further set out particular embodiments of the disclosure.

### REFERENCES

[1] M. M. Maciel, T. R. Correia, M. Henriques, J. F. Mano, Microparticles orchestrating cell fate in bottom-up approaches, Curr Opin Biotechnol 73 (2022) 276-281.
[2] N. R. Labriola, A. Azagury, R. Gutierrez, E. Mathiowitz, E. M. Darling, Concise Review: Fabrication, Customization, and Application of Cell Mimicking Microparticles in Stem Cell Science, Stem Cells Transl Med 7 (2) (2018) 232-240.
[3] M. H. Amer, M. Alvarez-Paino, J. McLaren, F. Pappalardo, S. Trujillo, J. Q. Wong, S. Shrestha, S. Abdelrazig, L. A. Stevens, J. B. Lee, D. H. Kim, C. Gonzalez-Garcia, D. Needham, M. Salmeron-Sanchez, K. M. Shakesheff, M. R. Alexander, C. Alexander, F. R. Rose, Designing topographically textured microparticles for induction and modulation of osteogenesis in mesenchymal stem cell engineering, Biomaterials 266 (2021) 120450.
[4] M. C. Gomes, J. F. Mano, Chemical modification strategies to prepare advanced protein-based biomaterials, Biomaterials and Biosystems 1 (2021) 100010.
[5] M. D. Neto, M. B. Oliveira, J. F. Mano, Microparticles in Contact with Cells: From Carriers to Multifunctional Tissue Modulators, Trends Biotechnol 37 (9) (2019) 1011-1028.
[6] K. K. Lai, R. Renneberg, W. C. Mak, Bioinspired Protein Microparticles Fabrication by Peptide Mediated Disulfide Interchange, RSC Advances 4 (2014) 11802-11810

1. A method for obtaining a protein microparticle from a platelet lysate comprising the following steps:
lyophilizing the platelet lysate;
dissolving the lyophilized platelet lysate in phosphate buffer saline to obtain a platelet lysate solution;
adding a reducing agent to the platelet lysate solution to obtain a reduced platelet lysate solution;
adding an oxidizing agent to the reduced platelet lysate solution to form the microparticles by precipitation.

2. The method of claim 1, wherein the lyophilized platelet lysate is dissolved in phosphate buffer saline at a concentration ranging from 10 to 20% (w/v).

3. The method of claim 1, wherein the reducing agent is selected from a list comprising mercaptoethanol, dithiothreitol, L-cysteine hydrochloride, cysteamine, and dithiobutylamine.

4. The method of claim 1, wherein the reducing agent is L-cysteine hydrochloride.

5. The method of claim 1, wherein the concentration of the reducing agent ranges from 1.5%-10% (w/v).

6. The method of claim 1, wherein the oxidizing agent is hydrogen peroxide.

7. The method of claim 1, wherein the concentration of the oxidizing agent ranges from 1%-5% (w/v).

8. A microparticle obtainable by the method of claim 1, comprising a plurality of protein from platelet lysate.

9. The microparticle of claim 8, comprising serum albumin, serotransferrin, keratin I, keratin II, or mixtures thereof.

10. The microparticle of claim 8, wherein the surface of the microparticle comprises a plurality of subunits, and thicknesses between 0.1 μm and 0.2 μm, and spaced apart between 0.5 μm to 4 μm.

11. The microparticle of claim 8, wherein the size of the microparticle ranges from 10 to 100 μm.

12. The microparticle of claim 8, wherein the surface of the microparticle is chemically modified with a chemical moiety.

13. A composition comprising the microparticle as described in claim 8.

14. The composition of claim 13, wherein the composition is an injectable composition.

15. The composition of claim 13, further comprising a plurality of cells, a therapeutic molecule, a suitable cell culture media, or mixtures thereof.

16. The microparticle of claim 8, wherein the microparticle is suitable as a scaffold for cell adhesion, cell proliferation and/or cell differentiation.

17. An article comprising the microparticle described in claim 8, wherein the article is a hydrogel, a fiber, a liquified capsule, a bioink, a microarray, or a lab-on-a-chip platform.

18. (canceled)

19. (canceled)

20. (canceled)

21. The composition of claim 13, wherein the composition is suitable for the treatment of a bone defect, cartilage abrasion or myocardial infarction.

22. The microparticle of claim 8, wherein the microparticle is suitable for the manufacture of a medicament for the treatment of a bone defect, cartilage abrasion or myocardial infarction.

23. A method for treating or preventing a bone defect, cartilage abrasion or myocardial infarction in a subject, the method comprising administering the microparticle of claim 8 to the subject.
